(19) United States
(12) Patent Application Publication
(10) Pub. No.: US 2005/0059083 A1
(43) Pub. Date: Mar. 17, 2005

(54) High throughput method to identify ligands for cell attachment

(75) Inventors: Andrea Liebmann-Vinson, Willow Springs, NC (US); Jonathan A. Rowley, Chapel Hill, NC (US); Chris H. Bodily, Syracuse, UT (US); Mohammad A. Heidaran, Cary, NC (US)

Correspondence Address: WILMER CUTLER PICKERING HALE AND DORR LLP, THE WILLARD OFFICE BUILDING, 1455 PENNSYLVANIA AVE, NW, WASHINGTON, DC, 20004, US

(73) Assignee: Becton Dickinson and Company

(21) Appl. No.: 10/662,640

(22) Filed: Sep. 15, 2003

(51) Int. Cl. G01N 33/53

(52) U.S. Cl. 435/7.1

(57) ABSTRACT

A high throughput method is provided for identifying agents capable of producing a desired biological response in whole cells. The method includes the steps of providing receptacles having a culture surface; placing different mixtures of single agents into selective ones of the receptacles according to a statistical design; and immobilizing the mixtures of single agents to the culture surface. The method further includes contacting the immobilized agents with the whole cells; and acquiring data which is indicative of a desired biological response in the contacted cells. The method also includes using statistical modeling of the acquired data to determine which mixtures of single agents and/or which single agents in these mixtures are effective in producing the desired biological response in the contacted cells.

### FIELD OF THE INVENTION

The present invention relates generally to the field of high throughput screening methods. In particular, the present invention relates to high throughput screening methods that can be used to identify mixtures of single agents and single agents within these mixtures that elicit a desired biological response in the cell.

### BACKGROUND OF THE INVENTION

It is known that attachment-dependent cells need a suitable culture substrate that allows for cell attachment in order to survive in vitro cell culture. Typically, proteins in media immobilize arbitrarily onto the surface of the cell culture substrate to form a layer to which cells can attach. The cell surface receptors, e.g., integrins, mediate cell attachment to such a protein layer, for example, by reacting with an extracellular matrix (ECM) protein such as fibronectin, that is present and still biologically active in this serum protein layer. Upon cell attachment to a surface through cell surface receptor-ligand interactions, internal signaling pathways are triggered within the cell, ultimately determining the fate of the cell, e.g., survival, proliferation, or differentiation. A disadvantage of using serum protein contained in the media to attach cells to the cell culture substrate is that, in contrast to in vivo biological processes, signaling pathways are triggered non-specifically and arbitrarily due to the non-specific and arbitrarily formed serum protein layer. Another disadvantage is that protein that is adsorbed onto the substrate from the media can be solubilized back into the media, and thus leave the surface, which further results in the substrate surface being poorly defined.

In other conventional cell culture systems, proteins to which cells can attach can be in the form of protein coatings that have been applied to the culture vessel prior to adding cells in cell culture media. Proteins that are adsorbed as the coating on the culture surface can be solubilized back into the culture medium and thus leave the culture surface.

For cells to be used in therapies to treat or cure diseases in humans, it is desirable to control cell fate, e.g., cell survival, proliferation and differentiation, when cells are maintained in culture in vitro. It is thus necessary to control cell surface receptor interaction with ligands present on the in vitro culture substrate. In order to gain control over cell-surface interactions, a suitable culture substrate, such as polystyrene, can be coated with a polymer which does not allow for cell attachment, even when serum proteins are used in the culture media. This coating thus eliminates the uncontrolled and arbitrary adsorption of the serum proteins. Biologically active ligands suitable to interact with cell surface receptors are then immobilized on this coating while maintaining the biological activity of the ligands. This concept is known. For example, it is known to use hyaluronic acid or algenic acid as a surface coating upon which the cell adhesion ligands can be immobilized using chemistries resulting in stable covalent bonds between the coating and the cell adhesion ligands. This prevents the cell adhesion ligand from being solubilized and leaving the surface. Moreover, the coating itself does not support cell adhesion. This is described in copending, commonly owned U.S. application Ser. No. 10/259,797, filed Sep. 30, 2002.

It is known to study one immobilized ligand and its effect on a certain cell type at a time. However, it is likely that mixtures of cell adhesion ligands and extrinsic factors are required in order to achieve a desired cell fate. A great number of cell adhesion ligands are known and used in cell adhesion studies. It can thus be a tedious task to find the right cell adhesion ligand or cell adhesion ligand combinations to place on a cell culture surface for optimal cell adhesion for a given cell type.

Therefore, there is a need in the art for higher throughput methods to identify cell adhesion ligands and/or extrinsic factors for a given cell type. This is of particular interest for cells that do not survive or only survive by drastically altering their differentiation state in conventional cell culture systems, a prime example being primary mammalian cells. In particular, there is a need in the art for a statistical experimental design that can be used to systematically explore the interactions between mixtures of factors that are required in order to achieve a desired fate for a given cell type.

### SUMMARY OF THE INVENTION

The present invention provides a high throughput method for identifying agents capable of producing a desired biological response in whole cells. In particular, the method includes the steps of providing receptacles having a culture surface; placing different mixtures of single agents into selective ones of the receptacles according to a statistical design; and immobilizing the mixtures of single agents to the culture surface. The method further includes contacting the immobilized agents with the whole cells; and acquiring data which is indicative of a desired biological response in the contacted cells. The method also includes using statistical modeling of the acquired data to determine which mixtures of single agents and/or which single agents in these mixtures are effective in producing the desired biological response in the contacted cells.

### BRIEF DESCRIPTION OF THE FIGURES

FIG. 1 is a schematic representation of preferred method steps used in the present invention.

FIG. 2 is a schematic representation of exemplary test wells.

FIG. 3 is a schematic representation of a 96-well plate layout comprising different mixtures of single agents. The layout is created using a statistical design in which generic factors in the design each represent a single agent.

FIG. 4 is a schematic representation of a 96-well plate layout comprising different mixtures of single agents. The layout is created using a statistical design in which generic factors in the design each represent a mixture of agents.

FIG. 5 is a schematic representation of a scenario that can be used in developing the statistical design of the method of the present invention.

FIG. 6 is a schematic representation of a further scenario that can be used in developing the statistical design of the method of the present invention.

FIG. 7 is a spreadsheet showing a mixture design for the layout of a 96-well plate developed using the scenario in FIG. 6, wherein the total fluid volume in a well is divided up based on the number of factors present.

FIG. 8 shows a 96-well plate layout created based on a statistical design of the spreadsheet in FIG. 7.

FIG. 9 is a fluorescent microscope image of fluorescently labeled cells attached to the wells of the 96-well plate with the layout shown in FIG. 8.

FIG. 10 is a graph of the nuclei count vs. well No. obtained following analysis of the microscope image in FIG. 9.

FIG. 11 is a graph of Ln (cell count-no serum+1) vs. deviation from the reference blend obtained using a mixture-model analysis of information from FIGS. 6-10.

FIG. 12 is a graph of Ln (cell count-10% serum+1) vs. deviation from the reference blend obtained using a mixture-model analysis of information from FIGS. 6-10.

FIG. 13 is a spreadsheet showing a Plackett-Burman statistical design for the layout of a 96-well plate.

FIG. 14 shows the identity of the factors in the statistical design in FIG. 13.

### DETAILED DESCRIPTION OF THE INVENTION

As defined herein, “agents” are growth effector molecules that bind to cells and regulate the survival, differentiation, proliferation or maturation of target cells or tissue. Examples of suitable agents for use in the present invention include growth factors, extracellular matrix molecules, peptides, hormones and cytokines.

The term “agent-immobilizing material” is defined herein as a biocompatible polymer that can serve as a link between the culture surface and an agent.

As defined herein, the term “immobilize,” “immobilized,” and the like is to render an agent(s), i.e., growth effector molecules, immobile on a culture surface, such as a well surface or the surface of a scaffold contained within a well. This term is intended to encompass passive adsorption of the agents to the culture surface, as well as direct or indirect covalent attachment of the agents to the culture surface.

“Factors” are the names of the variables in the experiment, and represent the things that the experiment changes from one trial or run (for e.g., one well) to the next. In the present invention, “factor” is a generic name for a single agent or mixture of single agents. Factors are combined according to a statistical design to form different mixtures in the experiment.

“Statistical Design”, as defined herein is an experimental design that assists the user in finding a combination of adjustable variables (i.e., factors) to produce the best experimental outcome, dramatically reducing the number of experiments needed to achieve that objective. In the present invention, a suitable statistical design is generated using generic factor names which represent the agents being tested. The design includes factor levels that can be the amounts and/or concentrations of the factors or that can be converted to the actual amounts and/or concentrations of the factors. The design also includes experimental runs, which are numbered. Experimental runs specify the combinations of factors and the levels thereof to test, and each corresponds to a single well on a multiwell plate, for example. The experimental runs can be mapped to wells on a generic multiwell plate.

As used herein, the terms “pre-treatment” and “pre-treated” refers to the addition to a surface or other substrate of functional groups which are chemically involved in the covalent bond subsequently formed with the agent-immobilizing material (i.e., a biocompatible polymer). For example, a surface of a microtitre well can be subjected to amino-plasma treatment to create an amine-rich surface onto which the agent-immobilizing material may be coupled.

As described above, the present invention relates to a high throughput method for identifying agents capable of producing a desired biological response in whole cells. This method includes the steps of: providing receptacles having a culture surface; placing different mixtures of single agents into selective ones of the receptacles according to a statistical design; immobilizing the mixtures of single agents to the culture surface; and contacting the immobilized agents with the whole cells. The method further includes acquiring data indicative of the desired biological response in the contacted cells; and determining which mixtures of the single agents and/or which of the single agents in these mixtures are effective in producing the desired biological response using statistical modeling of the acquired data. In one desired embodiment, the desired biological response may be selected from the following: cell adhesion, cell survival, cell differentiation, cell maturation, cell proliferation and combinations thereof.

As described above, it is likely that mixtures of single agents are required in order to achieve a desired cell fate. A great number of growth effectors are known. For example, growth effector molecules that bind to cell surface receptors or are taken up through ion channels or transports and regulate the survival, differentiation, proliferation or maturation of these cells include growth factors, extracellular matrix molecules, peptides, hormones and cytokines, of which there are many examples. It can therefore be a tedious task to find the right growth effector or growth effector combinations to place on a cell culture surface to achieve a desired cell fate for a given cell type.

The present invention solves a need in the art by providing for higher throughput methods to identify mixtures of agents that elicit a desired biological response for a given cell type.

In preferred embodiments of the method of the present invention, mixtures of single agents are covalently immobilized to an agent-immobilizing material on a culture surface, such as the receptacle surface. It is also well within the contemplation of the present invention that mixtures of single agents can be passively adsorbed onto the culture surface. The culture surface to which the agents are immobilized can also be a scaffold contained within the receptacle.

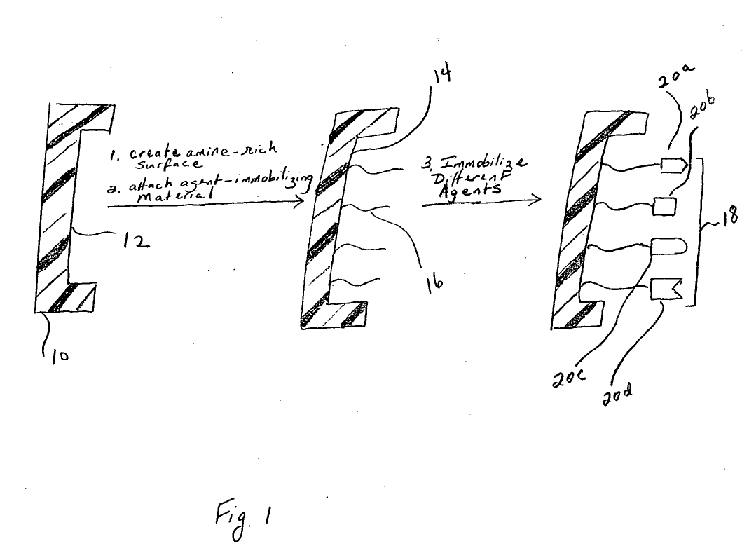

Referring now to FIG. 1, a preferred embodiment is shown wherein receptacle 10 is provided with a surface 12 which can be amino-plasma treated so as to create aminated surface 14 onto which agent-immobilizing material 16 can be attached. As will be described in further detail below, agent-immobilizing material 16 is preferably a biocompatible polymer which has been coupled to aminated surface 14. Mixtures 18 of single agents 20, e.g., 20a-d are desirably covalently immobilized to agent-immobilizing material 16.

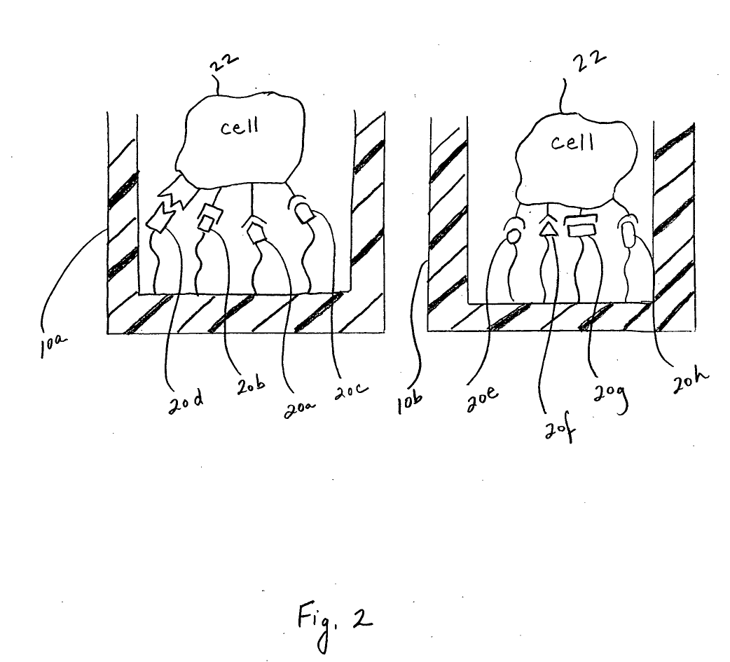

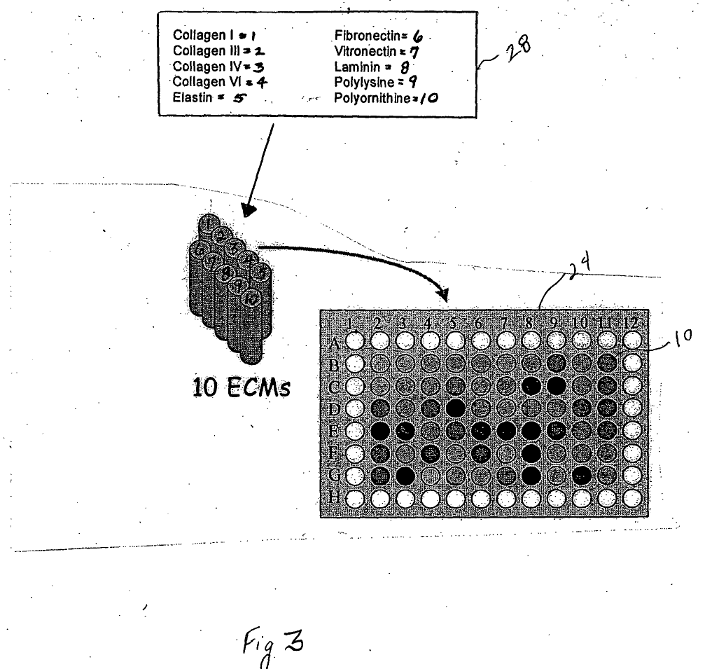

With reference now to FIG. 2, according to the present invention, different mixtures of single agents are placed into the receptacles according to a statistical design, which will be described in greater detail below. As shown in FIG. 2, the composition of agents 20a-d in receptacle 10a is different from that in a second receptacle 10b, where the composition comprises single agents 20e-h. It is noted, however, that more than one receptacle can include the same agent. For example, it is well within the contemplation of the present invention that a given agent may have a positive effect on achieving a desired cell fate when surrounded by a certain combination of other agents, and that this same agent may have a neutral effect or no effect on achieving a desired cell fate when surrounded by a different combination of agents, Therefore, it would be of benefit to provide an agent in different compositions with other agents to assess these effects. Referring again to FIG. 2, once agents 20 have been placed as different mixtures into the various receptacles 10 according to a statistical design, these mixtures 18 are contacted with whole cells 22. Agents 20 bind to cells 22 and are capable of producing one or more of the desired biological response in the contacted cells. A determination as to the effectiveness of a given mixture of agents or of single agents within the mixture at eliciting the desired response in the cell-type is ascertained based on acquired experimental data. Said data can be acquired using methods including, but not limited to, immunocytochemistry analysis, microscopy or functional assays.

Referring now to FIGS. 3-6, aspects of the statistical design will now be described in further detail. Referring in particular to FIG. 3, receptacles 10 are shown which correspond to the wells of the 96-well plate 24. This 96-well plate is comprised of rows A-H and columns 1-12. As shown in FIG. 3. it is one aspect of the present invention that the identity of single agents 20 or mixtures 18 in FIGS. 1 and 2 are represented by generic factor names. The factors are the variables in the experiment.

For example, as shown in FIG. 3, generic factors 1-10 are representative of the ten single extracellular matrix proteins indicated in box 28. In this example, generic factor 1 is Collagen I, generic factor 2 is Collagen III, etc. Each of these factors can be combined with one or more of the other factors to generate mixtures for the plate layout.

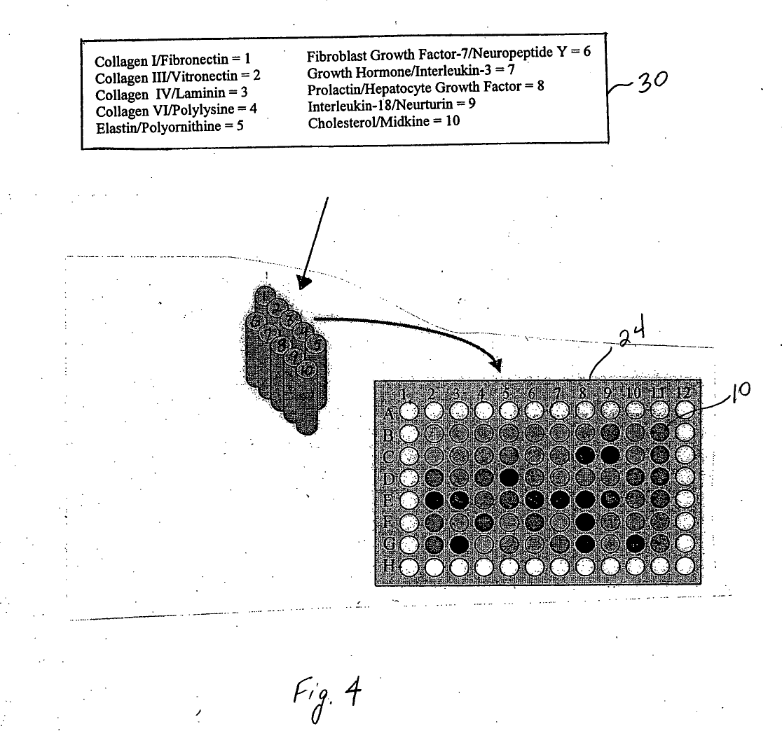

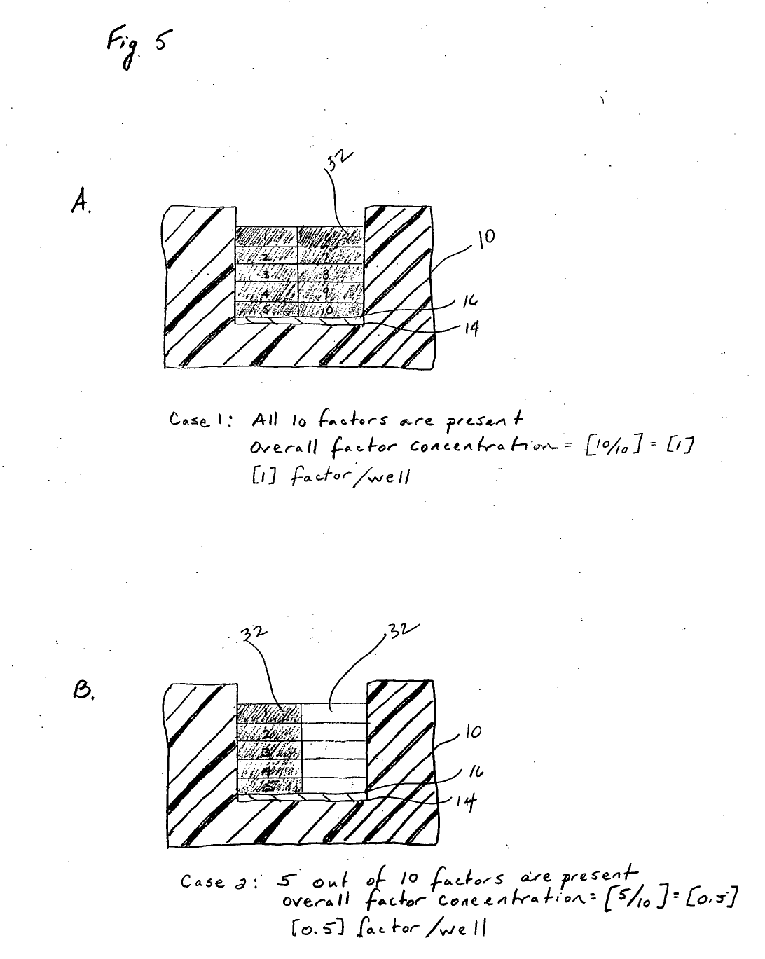

With reference now to FIG. 4, it is also well within the contemplation of the present invention that these generic factors 1-10 may each represent more than one agent. For example, as indicated in box 30, generic factor 1 in this example is representative of a mixture of Collagen I and Fibronectin; generic factor 2 is representative of a mixture of Collagen III and Vitronectin, etc. Each of these generic factors can similarly be combined with other generic factors to generate complex mixtures for the plate layout.

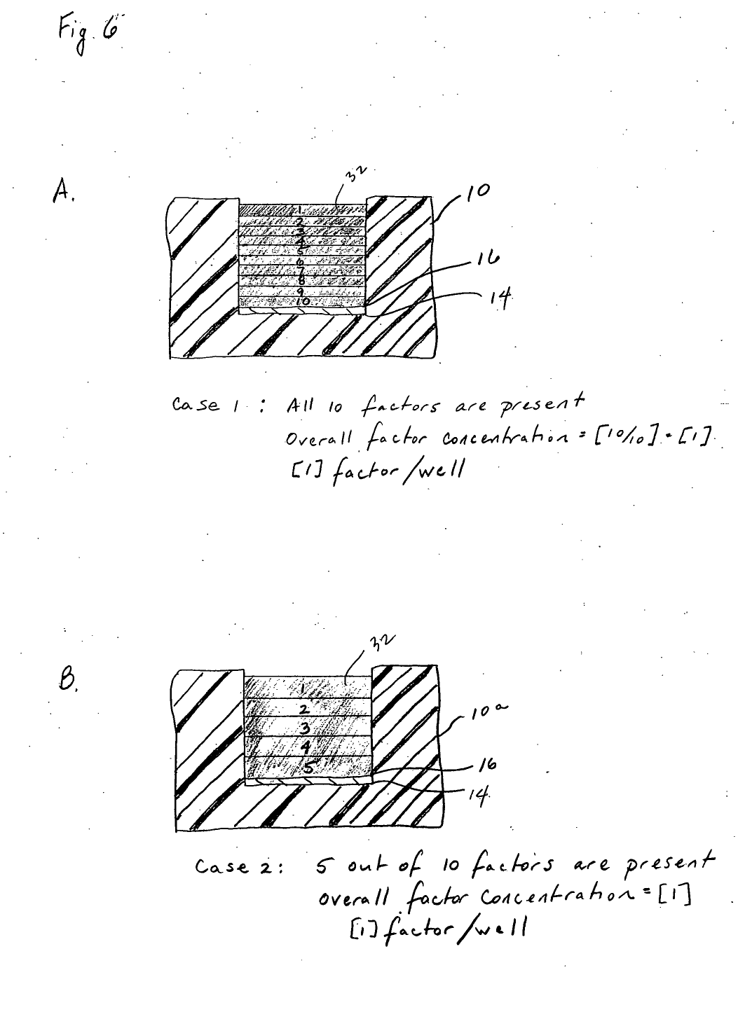

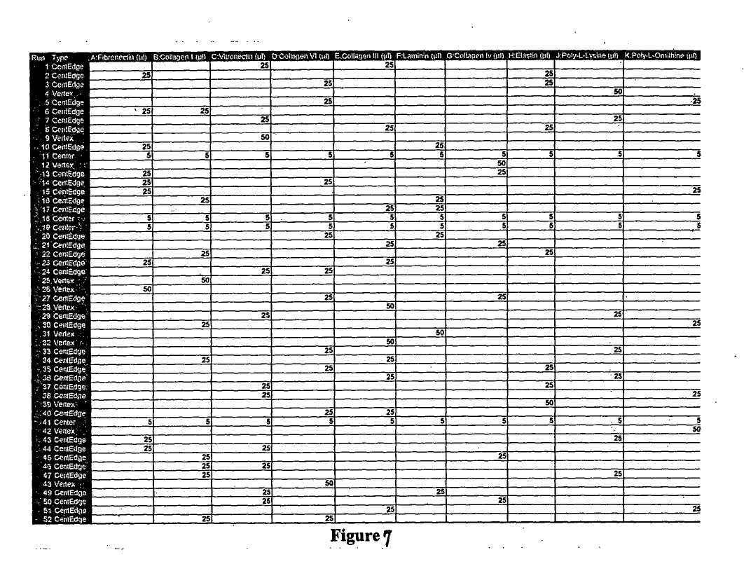

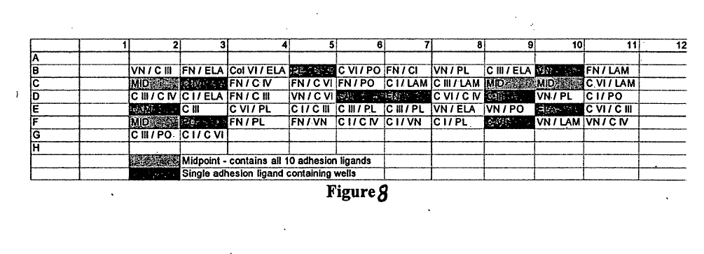

FIGS. 5 and 6 will now be described with reference to the embodiment shown in FIG. 3, wherein each of generic factors 1-10 corresponds to a single agent at a given concentration.

As shown schematically in FIG. 5, a scenario is presented in which the total fluid volume within receptacle 10 is divided into ten equal volume compartments 32. Each well of a 96-well plate may contain all ten factors (e.g., single agents) or a subset of these factors. As shown in FIG. 5a, in case 1, all ten factors are present and all ten factors occupy a fluid compartment 32. The overall factor concentration in well 10 shown in FIG. 5a, is [10/10]=[1]. This provides an overall concentration of factor equivalent to [1] per well. FIG. 5b represents a different well on the same 96-well plate, for example. In this situation (case 2), only five out of the ten factors are present. Again, the fluid volume is divided into ten equal compartments 32. In case 2, when a factor is present, the fluid compartment is filled with the factor. However, in case 2, five out of the ten volume compartments are not filled with a factor, but are rather filled with a “place holder”, such as media. In case 2 of FIG. 5b, the overall factor concentration equals [0.5]. Therefore, the overall factor concentration in the wells shown in FIG. 5b is [0.5] factor per well. The overall factor concentration in case 1 is not equivalent to the overall factor concentration in case 2. Therefore, in one embodiment of the present invention, the total concentration of the agents in each receptacle can be different. Moreover, in both case 1 and case 2, the concentration of a single factor is the same between wells. For example, the concentration of factor 1, which can represent a single Collagen I ligand is the same between the wells.

With reference now to FIG. 6, another scenario is presented wherein specific consideration is given to the surface chemistry requirements. In particular, in this scenario the overall density of factor is kept constant from well to well and only the factor composition is allowed to change between wells. In other words, the concentration of a factor can be different from well to well, but each well has the same amount of factor immobilized overall. As shown in FIG. 6, the total fluid volume present in a given well is divided up based on the number of factors present. Again, for the sake of simplicity, we can assume that one factor corresponds to one single agent, although the present invention is not limited to this situation. As shown in FIG. 6a, all ten factors are present and the overall factor concentration equals [10/10]=[1] for an overall factor concentration of [1] factor per well. In FIG. 6b, only five out of the ten factors are present, but the fluid volume 32 of each of these five factors is two times that of the volumes 32 of each of the factors shown in FIG. 6a. Consequently, the overall factor concentration shown in FIG. 6b is the same as that shown in FIG. 6a for a total concentration of [1] factor per well. Therefore, in one embodiment of the present invention, the total concentration of the agents in each receptacle is the same. Based on FIG. 6, it can be seen that whereas the overall factor concentration is constant between the well shown in 6a and the well shown in 6b, the concentration of a single factor can be different between these wells. In particular, with reference to factor 1, which may be representative of Collagen I, the concentration of this single agent in FIG. 6b would be twice that shown in FIG. 6a. Therefore, in a further embodiment of the present invention, the concentration of an individual agent differs between the receptacles.

It is noted that each of the scenarios depicted in FIGS. 5 and 6 are feasible and can be used for screening cell adhesion ligands, but the statistically designed experiment presented in the example section below was developed using the scenario shown in FIG. 6.

The present invention provides for methods which use a format, such as a 96-well plate format, to screen a plurality of different mixtures of agents in parallel for their ability to elicit a desired response in a cell. In one embodiment, the method involves placing different mixtures of agents into selective wells of a multi-well plate according to a statistical design. The method may further include the step of placing single agents into other of the wells. The agents are subsequently immobilized to a culture surface, such as a well surface. The method also includes delivering a fluid sample comprising a cell-type to the wells. After an appropriate incubation time between the cells and the samples in the various wells, evidence of an interaction between the cells and the well components can be detected, either directly or indirectly. For example, data can be acquired using functional assays, immunocytochemistry, or microscopy.

Suitable statistical designs for use with the present invention include, but are not limited, to the following: fractional factorial design, D-optimal design, mixture design and Plackett-Burman design. In one preferred embodiment, the statistical design is a mixture design. In another embodiment, the design is a space-filling design based on a coverage criteria, a lattice design, or a latin square design.

In desired embodiments, the culture surface, which may be pre-treated, is coated with an agent-immobilizing material. The agent-immobilizing material is desirably a biocompatible polymer which does not support cell adhesion and which can serve as a flexible link (tether) between the culture surface and the agents. Examples of suitable polymers include synthetic polymers like polyethylene oxide (PEO), polyvinyl alcohol, polyhydroxylethyl methacrylate, polyacrylamide, and natural polymers such as hyaluronic acid and algenic acid.

In desired embodiments, culture surfaces are selected from, but not limited to, the following: polystyrenes, polyethylene vinyl acetates, polypropylene, polymethacrylate, polyacrylates, polyethylenes, polyethylene oxide, glass, polysilicates, polycarbonates, polytetrafluoroethylene, fluorocarbons, and nylon. It is also well within the contemplation of the present invention that the culture substrates may wholly or partially include biodegradable materials such as polyanhydrides, polyglycolic acid, polyhydroxy acids such as polylactic acid, polyglycolic acid and polylactic acid-glycolic acid copolymers, polyorthoesters, polyhydroxybutyrate, polyphosphazenes, polypropyl fumurate, and biodegradable polyurethanes.

The culture surface to which the agents can be adsorbed or tethered can be pre-treated. For example, cell culture surfaces bearing primary amines can be prepared by plasma discharge treatment of polymers in an ammonia environment. The agent-immobilizing material can then be covalently attached to these aminated surfaces using standard immobilization chemistries, as described in copending, commonly owned U.S. application Ser. No. 10/259,797, filed Sep. 30, 2002, the entire contents of which are incorporated herein by reference. Two processes used commercially to create tissue culture treated polystyrene are atmospheric plasma treatment (also known as corona discharge) and vacuum plasma treatment, each of which is well known in the art. Plasmas are highly reactive mixtures of gaseous ions and free radicals. An amino-plasma treatment or oxygen/nitrogen plasma treatment can be used to create an amine-rich surface onto which biocompatible polymers such as hyaluronic acid (HA) or algenic acid (AA) may be coupled through carboxyl-groups using carbodiimide bioconjugate chemistries, as described in U.S. application Ser. No. 10/259,797. The resulting surfaces will not allow cells to attach, even in the presence of high, e.g., 10-20% serum protein concentrations present in the cell culture media. An example of pre-treated tissue culture polystyrene products that can be used to covalently link the agent via the agent-immobilizing material are the PRIMARIA™ tissue culture products (Becton Dickinson Labware), which are created using oxygen-nitrogen plasma treatment of polystyrene and which result in the incorporation of oxygen- and nitrogen-containing functional groups, such as amino and amide groups.

Agents, such as extracellular matrix proteins, peptides, etc. can be subsequently covalently coupled to the HA or AA surface described above utilizing the amine groups on the proteins/peptides and either the carboxyl groups on the HA or AA, or aldehyde groups created on the HA or AA by oxidation using sodium periodate, for example.

For example, the terminal sugar of human placental hyaluronic acid can be activated by the periodate procedure described in E. Junowicz and S. Charm, “The Derivatization of Oxidized Polysaccharides for Protein Immobilization and Affinity Chromotography,” Biochimica et. Biophysica Acta, Vol. 428: 157-165 (1976), incorporated herein by reference. This procedure entails adding sodium or potassium periodate to a solution of hyaluranic acid, thus activating the terminal sugar which can be chemically cross-linked to a free amino group on an agent, such as the terminal amino group on an extracellular matrix protein. In another preferred embodiment, free carboxyl groups on the biocompatible polymer (for example, HA or AA) may be chemically cross-linked to a free amino group on the agent using carbodiimide as a cross-linker agent. Other standard immobilization chemistries are known by those of skill in the art and can be used to join the culture surfaces to the biocompatible polymers and to join the biocompatible polymers to the agents. For example, see “Protein Immobilization: Fundamentals and Applications” Richard F. Taylor, Ed. (M. Dekker, NY, 1991) or copending U.S. application Ser. No. 10/259,797, filed Sep. 30, 2002.

It is noted that whereas the tethering of the agents to aminated tissue culture surfaces via biocompatible polymers comprises one embodiment of the present invention, these agents can also be tethered via biocompatible polymers to carboxylated surfaces or hydroxylated surfaces using standard immobilization chemistries. Examples of attachment agents are cyanogen bromide, succinimide, aldehydes, tosyl chloride, avidin-biotin, photocrosslinkable agents, epoxides and maleimides.

As described above, it is an aspect of the present invention that mixtures of agents are contained within selective ones of the receptacles. Moreover, it is a further aspect of the present invention that other receptacles may contain a single agent. These agents may be tethered alone or in combinations to pre-treated tissue culture surfaces. The agents may be combined in any desired proportions. The relative amounts of different agents present on the culture surfaces can be controlled, for example, by the concentration of the agents in a coating composition. Alternatively, the loading density can be controlled by adjusting the capacity of the biocompatible polymers bound to the culture surface. This can be accomplished by, for example, controlling the number of reactive groups on the polymers that can react with the agents or by controlling the density of the biocompatible polymer molecules on the culture surface. Moreover, the agents can first be separately linked to the biocompatible polymers (tethers), and then the “loaded” tethers can be mixed in the desired proportions, and attached to the pre-treated substrate.

As described above, it is preferred that the agents are covalently immobilized via biocompatible polymers to a pre-treated tissue culture surface, which is desirably amine-rich. However, it is noted that it is well within the contemplation of the present invention that rather than covalently immobilizing the agents to the surfaces in this way, the agents can be immobilized to the culture surface (e.g., well surface) by passively adsorbing the agents to the surface. It is also well within the contemplation of the present invention that the agents can be immobilized on or impregnated within a scaffold, which can be placed in the receptacle and contacted with fluid containing the cells. Suitable scaffolds for use in the present invention and methods for immobilizing agents thereto or therewithin are described in copending, commonly owned U.S. application Ser. No. 10/259,817, filed Sep. 30, 2002, the entire contents of which are incorporated herein by reference.

Receptacles for use in the present invention can take any usual form, but are desirably tissue culture dishes, multi-well plates, flasks, tubes, and roller bottles. Configurations such as microtitre wells and tubes are particularly useful in the present invention and allow the simultaneous assay of a large number of samples to be performed manually in an efficient and convenient way. The assay can also be automated using, for example, microtitre wells and is capable of extensive automation because of automatic pipetters and plate readers. Other solid phases, particularly other plastic solid supports, may also be used.

It is noted that the method steps of the present invention can be readily automated. This is particularly so with microtitre plates as the format. Therefore, in one embodiment of the present invention, the receptacles can comprise the wells of a 96-well microtitre plate. Automatic pipetting equipment (for reagent addition and washing steps) and color readers already exist for microtitre plates. An example of an automated device for carrying out the present invention can include: a pipetting station and a detection apparatus, the pipetting station being capable of performing sequential operations of adding and removing reagents to the wells at specific time points in a thermostatic environment (i.e., temperature controlled environment).

As described above, agents for use in the present invention are growth effector molecules that bind receptors on the cell surface or are taken up through ion channels or transports and regulate the growth, replication or differentiation of target cells or tissue. In one embodiment, these agents are cell adhesion ligands and/or extrinsic factors. In desired embodiments, the agents can be extracellular matrix proteins, extracellular matrix protein fragments, peptides, growth factors, cytokines, and combinations thereof.

Preferred agents are growth factors and extracellular matrix molecules. Examples of growth factors include, but are not limited to, vascular endothelial-derived growth factor (VEGF), epidermal growth factor (EGF), platelet-derived growth factor (PDGF), transforming growth factors (TGFα, TGFβ), hepatocyte growth factor, heparin binding factor, insulin-like growth factor I or II, fibroblast growth factor, erythropoietin nerve growth factor, bone morphogenic proteins, muscle morphogenic proteins, and other factors known to those skilled in the art. Other suitable growth factors are described in “Peptide Growth Factors and Their Receptors I” M. B. Sporn and A. B. Roberts, Eds. (Springer-Verlag, NY, 1990), for example.

Growth factors can be isolated from tissues using methods known in the art. For example, growth factors can be isolated from tissue or can be produced by recombinant means. For example, EGF can be isolated from the submaxillary glands of mice and Genentech (South San Francisco, Calif.) produces TGF-β recombinantly. Other growth factors are also available from vendors, such as Sigma Chemical Co. (St. Louis, Mo.), R&D Systems (Minneapolis, Minn.), BD Biosciences (San Jose, Calif.), and Invitrogen Corporation (Carlsbad, Calif.), in both natural and recombinant forms.

Examples of suitable extracellular matrix molecules for use in the present invention include vitronectin, tenascin, thrombospondin, fibronectin, laminin, collagens, and proteoglycans. Other extracellular matrix molecules are described in Kleinman et al., “Use of Extracellular Matrix Components for Cell Culture,” Analytical Biochemistry 166: 1-13 (1987), or known to those skilled in the art.

Additional agents useful in the present invention include cytokines, such as the interleukins and GM-colony stimulating factor, and hormones, such as insulin. These are described in the literature and are commercially available.

Cells for use with the present invention can be any cells that can potentially respond to the agents or that need the agents for growth. For example, cells can be obtained from established cells lines or separated from isolated tissue. Suitable cells include most epithelial and endothelial cell types, for example, parenchymal cells, such as hepatocytes, pancreatic islet cells, fibroblasts, chondrocytes, osteoblasts, exocrine cells, cells of intestinal origin, bile duct cells, parathyroid cells, thyroid cells, cells of the adrenal-hypothalamic-pituitary access, heart muscle cells, kidney epithelial cells, kidney tubular cells, kidney basement membrane cells, nerve cells, blood vessel cells, cells forming bone and cartilage, and smooth and skeletal muscles. Other useful cells can include stem cells which may undergo a change in phenotypes in response to a select mixture of agents. Further suitable cells include blood cells, umbilical cord blood-derived cells, umbilical cord blood-derived stem cells, umbilical cord blood-derived progenitor cells, umbilical cord-derived cells, placenta-derived cells, bone marrow-derived cells, and cells from amniotic fluid. The cells can be genetically engineered. In preferred embodiments, the cells are cultured with agents which are tethered via a biocompatible polymer to a culture substrate, such as a well surface(s) of a 96-well microtitre plate. These cells can be cultured using any of the numerous well known cell culture techniques, such as those described in Freshney, “Cell Culture, A Manual of Basic Technique” 3^{rd} Edition (Wiley-Liss, NY, 1994). Other cell culture media and techniques are well known to those skilled in the art and can be used in the present invention.

Statistically designed experiments in accordance with the present invention will now be described.

### EXAMPLES

### Example 1

### Coupling of Hyaluronic Acid to an Amine-Rich Tissue Culture Surface

An oxygen/nitrogen plasma is used by Becton Dickinson Labware to create PRIMARIA™ tissue culture products. In particular, oxygen/nitrogen plasma treatment of polystyrene products results in incorporation of oxygen- and nitrogen-containing functional groups, such as amino and amide groups. For this experiment, HA was coupled to the amine-rich surface on PRIMARIA™ multi-well plates through carboxyl groups on HA using carbodiimide bioconjugates chemistries well known in the art, such as those described in “Protein Immobilization: Fundamentals and Applications” Richard S. Taylor, Ed. (M. Dekker, NY, 1991) or as described in copending U.S. application Ser. No. 10/259,797, filed Sep. 30, 2002.

### Example 2

### Coupling of ECM Proteins to Hyaluronic Acid

ECM agents were covalently attached to the HA polymer tethered to the culture surface. In particular, aldehyde groups were created on HA by oxidation using the periodate procedure described in E. Junowicz and S. Charm, “The Derivatization of Oxidized Polysaccharides for Protein Immobilization and Affinity Chromotography,” Biochimica et. Biophysica Acta, Vol. 428: 157-165 (1976). This procedure entailed adding sodium periodate to a solution of HA, thus activating the terminal sugar. Subsequently, the activated HA was coupled to the amine groups on the ECM proteins using standard immobilization chemistries, such as those described in “Protein Immobilization: Fundamentals and Applications” Richard F. Taylor, Ed. (M. Dekker, NY, 1991) or copending U.S. application Ser. No. 10/259,797, filed Sep. 30, 2002.

### Example 3

### Use of a Statistically Designed Experiment (Mixture Design) to Screen 10 Different ECM Proteins Simultaneously

In the present example, the statistical design is a mixture design. This design was used to identify pairs of factors, or single factors that had a positive effect on a cell response, and allows us to look at interactions between two ECMs. In this example, 10 single ECMs, each representing a single “factor” are used to created ECM mixtures for placement into the wells of a 96-well plate as shown in FIG. 3. The ECMs covalently attach to biocompatible polymers on the culture surface (see Examples 1 and 2). It is noted that without a statistical design for the experiment, it would take 2¹⁰ (1024) single experiments, or eleven 96-well plates, to test each of the 10 ECMs together with the others against a given cell-type.

In this example, a group of 10 adhesion ligands was selected and a 96-well plate was chosen as the format for this screen. To eliminate border effects due to uneven evaporation, only the inner 60 wells of the 96-well plate are to be used for the experiment. Wells in the outer rows and columns of the plate can thus be used for suitable controls.

The following 10 adhesion ligands were selected based on their common use as cell culture reagents, commercial availability and price: Collagen I (CI), Collagen III (CIII), Collagen IV (CIV), Collagen VI (CVI), elastin (ELA), fibronectin (FN), vitronectin (VN), laminin (LAM), polylysine (PL), and polyornithine (PO).

A statistical design was developed with special consideration of the surface chemistry requirements. In particular, in this experiment the scenario shown in FIG. 6 was used, wherein the overall adhesion ligand density was kept constant from well to well and only the adhesion ligand composition was allowed to change. In other words, the concentration of a single adhesion ligand could be different from well to well, but each well has the same amount of adhesion ligand immobilized overall. This scenario is further described above. An example of such design is shown in the spreadsheet in FIG. 7. The top row in FIG. 7 lists the 10 cell adhesion ligands used in this particular screen. The first column is a list of the experimental points that translate into a well in the 96-well plate, e.g., 52 wells in this case. The numbers in the spreadsheet are the actual volumes (in μL) of factor that is added to a particular well. In this particular design, factors get added to the wells at three volumes, e.g., 5 μL, 25 μL, or 50 μL. The total well volume in this case is 50 μL. Thus, for wells where one factor is added at 50 μL, the final well composition will comprise a single adhesion ligand covalently immobilized on the well surface. Accordingly, if 25 μL of a factor is added to a well, a second factor is added at 25 μL also, and the final well composition will comprise a mixture of two different cell adhesion ligands covalently immobilized on the well surface. When 5 μL of a factor are added, nine other factors are added at 5 μL each, as well, thus resulting in wells that comprise a mixture of all 10 cell adhesion ligands on the well surface. These experimental points containing all 10 adhesion ligands are called “mid points” and are an integral part of the statistical design in this example.

With reference now to FIG. 8, a 96-well plate layout is shown, which was translated from the particular statistical design shown in FIG. 7. In particular, the 96-well plate includes the well compositions indicated in FIG. 7, e.g., cell adhesion ligand combinations immobilized at the bottom of each well. In particular, the experimental runs in FIG. 7 correspond to rows/columns in FIG. 8, as follows: runs 1-10 in the design in FIG. 7 represent row B, columns 2-11, respectively on the plate layout in FIG. 8; runs 11-20 represent row C, columns 2-11; runs 21-30 represent row D, columns 2-11; runs 31-40 represent row E, columns 2-11; runs 41-50 represent row F, columns 2-11; and runs 51 and 52 represent row G, columns 2 and 3, respectively. As shown by the statistical design in FIG. 7 and the corresponding 96-well plate layout in FIG. 8, it is an embodiment of the present invention that, in addition to mixtures of agents, single agents can be placed in the receptacles.

### Example 4

### ECM Screen Specific to MC3T3-E1 Osteoblast Cells

MC3T3-E1 cells, originated from Dr. L. D. Quarles, Duke University, and were kindly provided by Dr. Gale Lester, University of North Carolina at Chapel Hill. These cells were grown using standard cell culture techniques. MC3T3-E1 is a well-characterized and rapidly growing osteoblast cell line that was chosen because it attaches aggressively to most commonly used tissue culture surfaces.

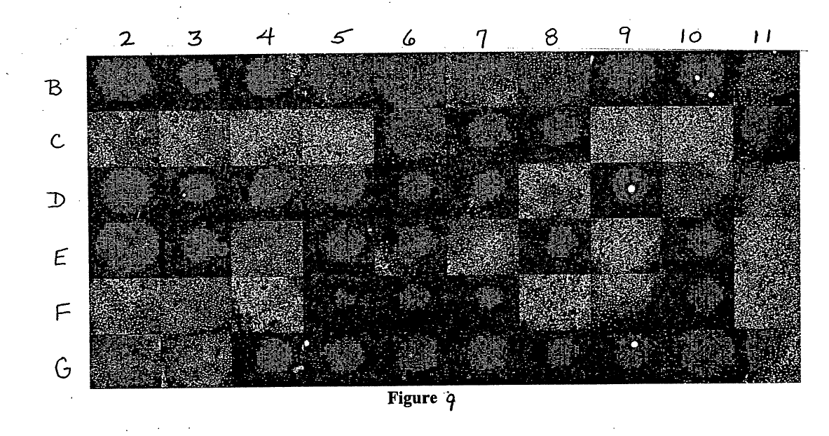

Cells were removed from cell culture flasks using trypsin-EDTA according to methods well known in the art. Cells were enumerated, spun down and resuspended in media containing no serum or, alternatively, in media containing 10% fetal calf serum. Cells were plated into the wells of a 96-well plate according to the layout shown in FIG. 8 and described in Example 3 above. The seeding density was about 10,000 cells per well. Cells were incubated on the plates overnight at 37° C. The following day, media and any cells not adhering to the immobilized agents on the well surfaces were removed. Any adhered cells were fixed by exposure to formalin for at least 15 minutes. Propidium iodite was used to fluorescently label the nuclei of said fixed adhered cells. A fluorescent microscope (Discovery-1, Universal Imaging Corporation, a subsidiary of Molecular Devices, Downingtown, Pa.) was used to acquire images of the fluorescently labeled cells attached to the wells in the ECM screening plate. An example of an image acquired from a 96-well plate is shown in FIG. 9. In particular, the layout is the same as that shown in FIG. 8, except that row G, column 4-11 are used as control wells. In FIG. 9, MC3T3-E1 cells in 10% fetal calf serum-containing media were placed into wells containing mixtures of agents that had been tethered to a hyaluronic acid surface, with the exception that wells G4-G9 contained a hyaluronic acid surface only and wells G10 and G11 comprised tissue culture grade polystyrene only. As expected, the hyaluronic acid surface only in wells G4-G9 prevented cell adhesion. Cell adhesion to the polystyrene surfaces in wells G10 and G11 was, in this example, surprisingly low. In contrast, some wells containing cell adhesion ligands showed strong cell adhesion, as can be seen by the large number of white spots, each of which represents the nucleus of an adhered cell.

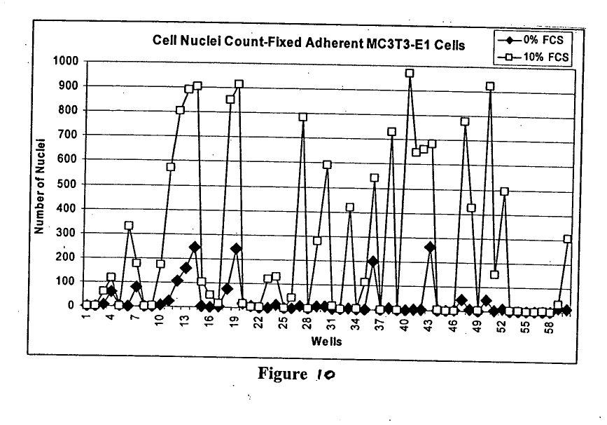

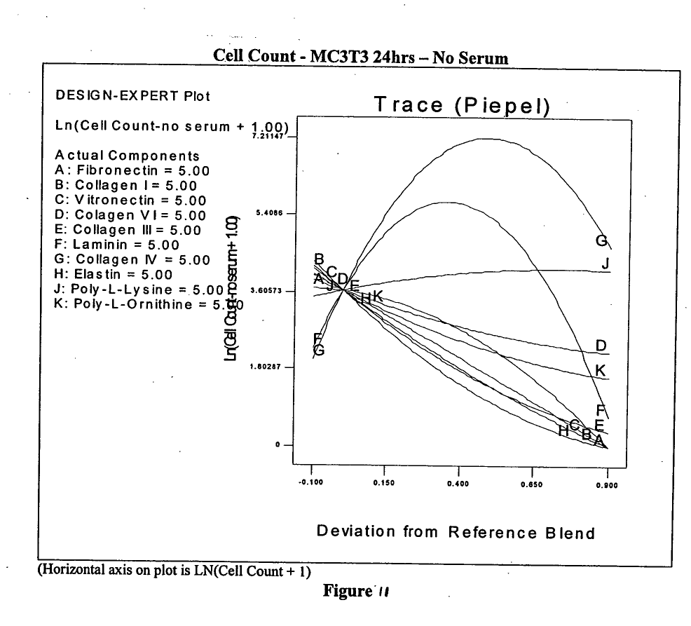

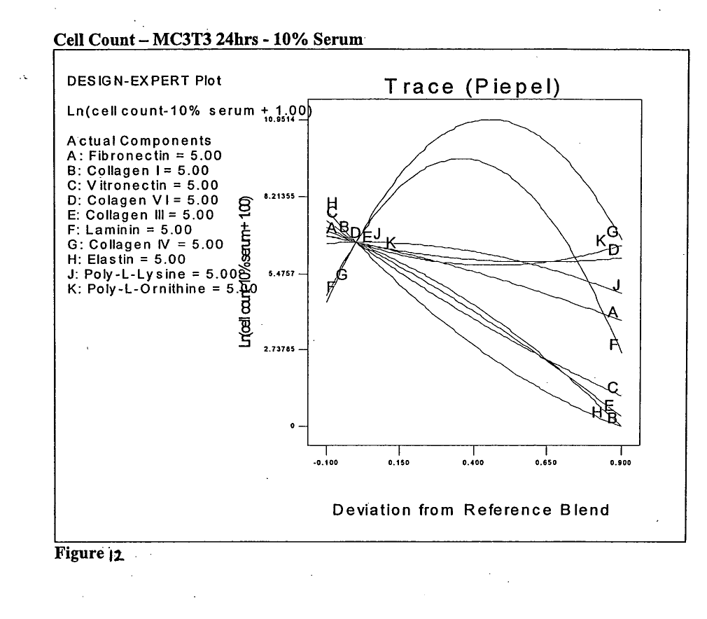

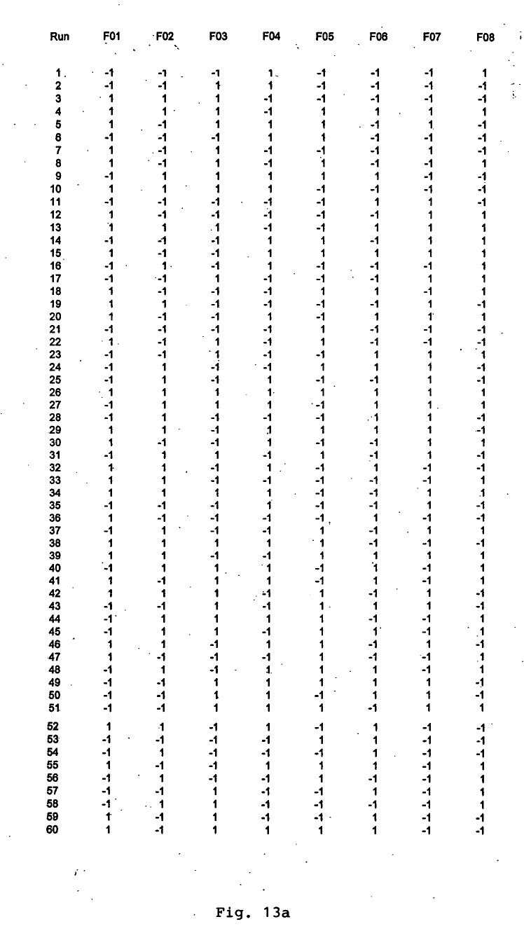

An image analysis software package (Meta Morph, Universal Imaging Corporation, a subsidiary of Molecular Devices, Downingtown, Pa.) was used to enumerate the fluorescently labeled cell nuclei in FIG. 9 and the nuclei count results for both cells in media containing no fetal calf serum and media containing 10% fetal calf serum are shown in FIG. 10. In FIG. 10, wells 1-10 correspond to row B, columns 2-11 in FIG. 9; wells 11-20 in FIG. 10 correspond to row C, columns 2-11 in FIG. 9, etc.

In FIG. 10, in the presence of 10% fetal calf serum, cell adhesion was observed for a number of wells. In the absence of serum, cell adhesion was reduced, but cell adhesion was still observed in a number of wells. In both cases, cell adhesion in some wells containing cell adhesion ligands according to the statistically designed experiment exceeded that of cells cultured on plain tissue-culture grade polystyrene (wells 59 and 60 in FIG. 9). The results obtained enabled the identification of a number of surfaces that support MC3T3-E1 adhesion better than tissue culture grade polystyrene, the most commonly used cell culture support.

In order to optimize the surfaces, one can follow two leads, e.g., the “best well” composition or the “best factors”. The determination of “best factors” is made following rigorous statistical analysis of the experimental results.

In the “best well” approach, the well with the best experimental outcome is chosen for further optimization. In the example shown in FIG. 10, one would choose well 40 (or well E11) which had the highest number of cell nuclei. This well contained a mixture of Collagen-type VI and Collagen-type III according to the plate layout shown in FIG. 8. The concentration of Collagen-type VI and Collagen-type III that was chosen for the immobilization step in the ECM screening plate preparation was based on initial concentration-dependent studies with the MC3T3-E1 cells using the model ECM, fibronectin. It is noted that a concentration which is optimal for one cell-type under investigation may not be optimal for another cell-type. Moreover, the concentration of a particular ECM which is optimal for a given cell type may not be the optimal concentration for another ECM, even when the same cell type is used. Similarly, the composition of a mixture in the “hit well” may not be optimal. For example, the surface of well E11, which was the “best well” comprised a 50/50 mixture of Collagen-type VI and Collagen-type III. Follow-up experiments may be performed to optimize the concentration of both ligands chosen for the immobilization step, as well as the composition of the mixture (a 50/50 mixture may not be the optimal composition) bound to the surface of a “hit” well for a given cell-type.

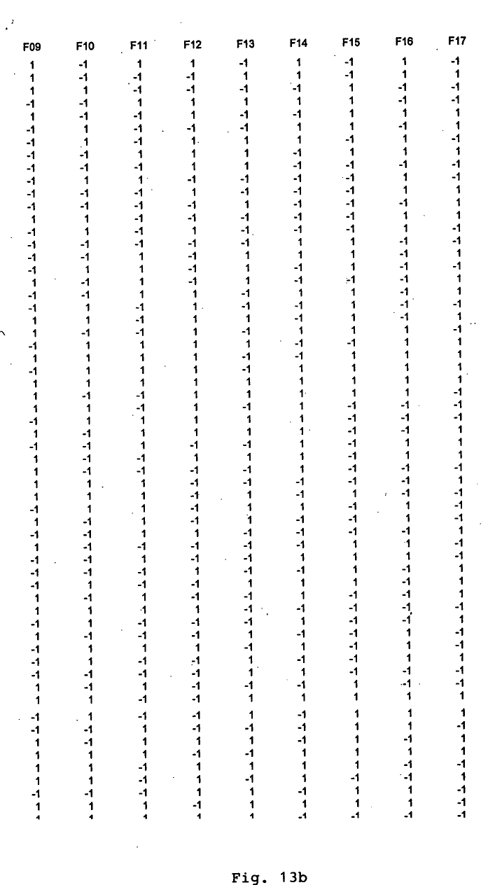

In the “best factors” approach, the experimental results are analyzed using statistical models. For the above-described example, a mixture-model analysis of the MC3T3-E1 data shows that Collagen IV, laminin, and poly-L-lysine (marginal effect) appear to increase the cell count when present at significant quantities with no serum as shown in FIG. 11. The points at which all the lines intersect correspond to mid-points, where all 10 ECMs were present at 5 μL each. This graph provides an indication as to how the cell count changes, depending on how far the well composition deviates from this reference “mid-point” blend. As can be seen, as the amount of Collagen IV or laminin increases, the cell counts increase.

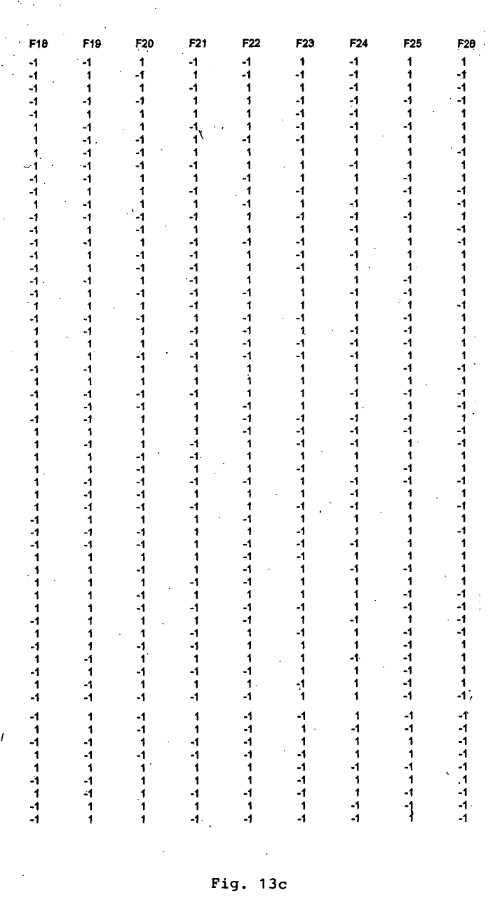

With reference now to FIG. 12, with 10% serum, any effect of poly-L-lysine that was seen in FIG. 11 diminishes, and only Collagen IV and laminin continue to show a positive effect on cell count.

It is noted that both the “best well” and “best factors” approaches are valid, but each approach can lead to different surface compositions. In the present example, the “best well” approach would lead to a surface comprising Collagen-type VI and Collagen-type III, while the “best factor” approach would lead to a surface comprising Collagen VI and laminin.

### Example 5

### Use of a Statistically Designed Experiment (Plackett-Burman Design) to Screen 30 Different Agents

Design

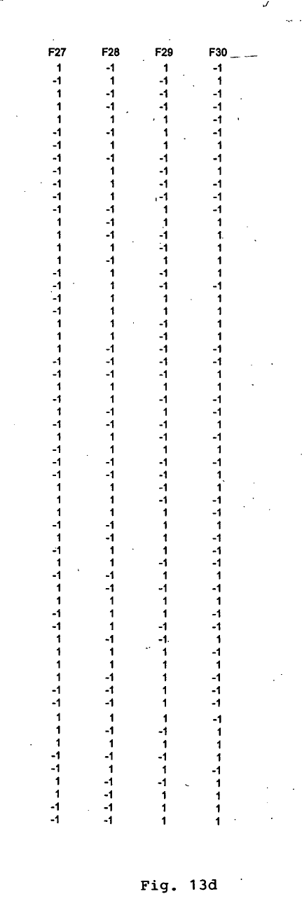

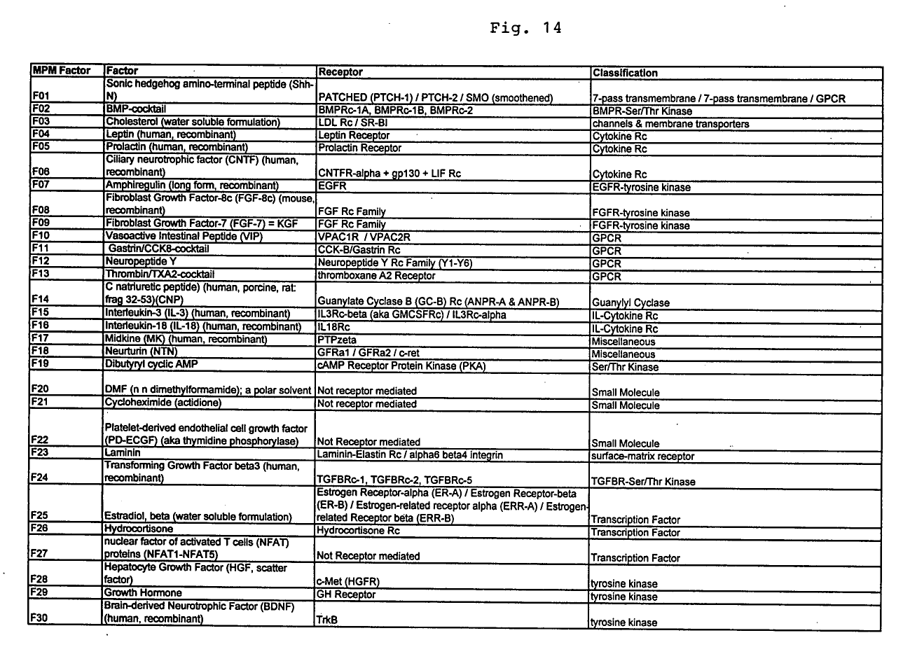

The present example describes a Plackett-Burman (PB) design as shown in FIG. 13(a-d), which was generated using a commercially available software package JMP™ from SAS Institute (Cary, N.C.). In particular, the screening design was generated using the custom design function in SAS/JMP V 4.0.5. The software package is a GUI oriented package, so there is no code to show. With reference to FIG. 13a, the first column is a list of the experimental points (runs) that translate into a well in the 96-well plate, e.g., 60 wells in this case. The numbers in the spreadsheet itself (−1 or 1) (FIGS. 13 a-d) is an indication of the level of a factor. In this example, “1” indicates the presence of the factor and “−1” indicates the absence of a factor. Moreover, in this example, if a factor is present in a given well, it is always at the same concentration in regard to the total volume of the well. The total concentration of agents may vary from well to well based on the number of agents included in the corresponding experimental run. The generic factor names are provided in the top row of FIGS. 13 a-d. FIG. 14 shows the identity of each of generic factors F01-F30 in the present experiment. For example, experimental run 1 in the first column may represent well 1 of a 96-well plate. From the statistical design shown in FIG. 13(a-d), it can be seen that the following factors are present (i.e., level “1”) in well 1: F04, F08, F09, F11, F12, F14, F16, F20, F23, F25, F26, F27, and F29.

Proposed Acquisition of Data and Statistical Analysis

Cells are plated into the wells of a 96-well plate in accordance with the design shown in the spreadsheet of FIG. 13(a-d). The seeding density is about 10,000 cells per well. Cells are incubated on the plates overnight at 37° C. The following day, media and any cells not adhering to the immobilized agents on the well surfaces are removed and any adhered cells are fixed by exposure to formalin for 15 minutes. The nuclei of the fixed adhered cells are fluorescently labeled and images are acquired with a fluorescent microscope as described above in Example 4. An image analysis software package (Meta Morph, Universal Imaging Corporation) is used to enumerate the fluorescently labeled cell nuclei and the nuclei count results for the cells are obtained. Based on these results, wells with the best experimental outcome (e.g., highest number of cell nuclei) are chosen for further optimization. By examining the contents of the wells that give the best results, information is gained regarding which factors and/or factor groups yields beneficial effects. By including many factors in the design, potentially more complex interactions between the factors can be determined. Follow-up screening experiments can focus on a particularly interesting factor combination discovered in the first round of screening.

Following the first screen, the main effects are estimated and reviewed. By “main effects”, it is meant the effect of a single agent acting independently. Interaction effects mean the combined effects of more than one single agent when the agents act in concert (not independently). At this point, relevant interactions among the agents typically are not estimated in the statistical model, but interactions among the agents would be expected to result in the best experimental runs, i.e., best wells. After the first round of screening, the best wells and the factors that are included in these wells (level=“1”) are identified. Follow-up experiments can be performed for each best well using all the factors included in the well, whether or not they had a positive, neutral, or negative effect in the preliminary statistical analysis. The experiments can be repeated with a subset of the agents identified in the best well so as to arrive at an optimum subset of factors for producing a desired response in a cell. Moreover, the experiment can be repeated, wherein the concentration of the agents in a best well are varied. Follow-up experiments can also be performed with the subset of single agents that had statistically significant main effects or by combining a subset of the best single agents with a subset of agents identified in the best mixtures.

It has been proposed that the control of cellular phenotypes via extracellular conditions is governed by high order interactions among the factors in the extracellular environment. The Plackett-Burman design presented here is believed to provide good statistical estimates of the main effects and also provides the opportunity to observe a diverse set of combinations of factors among its experimental runs. In this case, higher-order interactions would be expected to result in specific experimental runs as being “best wells” over and above what could be predicted by the individual main effects of the agents in the best wells.

1. A high throughput method for identifying agents capable of producing a desired biological response in whole cells, the method comprising the steps of:
(a) providing receptacles having a culture surface;
(b) placing different mixtures comprising single said agents into selective ones of said receptacles according to a statistical design;
(c) immobilizing said mixtures of single agents to said culture surface;
(d) contacting said agents from (c) with said whole cells;
(e) acquiring data indicative of said desired biological response in said contacted cells; and
(f) identifying which of said mixtures of single agents and/or which single agents in said mixtures are effective in producing said desired biological response in said contacted cells using statistical modeling of said acquired data.

2. The method of claim 1, further comprising the step of placing single said agents into others of said receptacles.

3. The method of claim 1, wherein said culture surface is coated with an agent-immobilizing material.

4. The method of claim 3, wherein said agent-immobilizing material is a biocompatible polymer selected from the group consisting of hyaluronic acid, algenic acid, polyethylene oxide, polyhydroxyethyl methacrylate, and combinations thereof.

5. The method of claim 3, wherein said agent-immobilizing material contains reactive groups for covalently immobilizing said agents.

6. The method of claim 3, wherein said agent-immobilizing material on said culture surface does not support cell adhesion.

7. The method of claim 1, wherein said agents are cell adhesion ligands and/or extrinsic factors.

8. The method of claim 7, wherein said agents are selected from the group consisting of extracellular matrix proteins, extracellular matrix protein fragments, peptides, growth factors, cytokines and combinations thereof.

9. The method of claim 1, wherein said data is acquired by immunocytochemistry analysis, microscopy, or functional assays.

10. The method of claim 1, wherein said desired biological response is selected from the group consisting of cell adhesion, cell survival, cell differentiation, cell maturation, cell proliferation and combinations thereof.

11. The method of claim 1, wherein said receptacles are wells of a 96-well plate.

12. The method of claim 1, wherein the total concentration of said agents in each receptacle is the same.

13. The method of claim 1, wherein the total concentration of said agents in each receptacle is different.

14. The method of claim 1, wherein the concentration of a single said agent differs between said receptacles.

15. The method of claim 1, wherein said statistical design is selected from the group consisting of a fractional factorial design, a d-optimal design, a mixture design and a Plackett-Burman design.

16. The method of claim 1, wherein said statistical design is a space-filling design based on a coverage criteria, a lattice design, or a latin square design.

17. The method of claim 1, further comprising repeating said steps with a subset of said identified mixtures of single agents.

18. The method of claim 1, further comprising repeating said steps, wherein the concentrations of agents in said identified mixtures are varied.

19. The method of claim 1, wherein said statistical modeling is an algorithm for comparing said acquired data with the statistical design.
